(19) United States
(12) Patent Application Publication
(10) Pub. No.: US 2003/0175827 A1
(43) Pub. Date: Sep. 18, 2003

(54) Stable thin film dried protein composition or device and related methods

(76) Inventors: Brett A. Stillman, Keene, NH (US); Weiwen Zhao, Nashua, NH (US); John L. Tonkinson, Keene, NH (US); Daniel S. Osborn, Sullivan, NH (US); Debra W. English, Jaffrey, NH (US)

Correspondence Address: Brian D. Voyce, 8401 Sterling Bridge Road, Chapel Hill, NC, 27516, US

(21) Appl. No.: 10/098,768

(22) Filed: Mar. 14, 2002

(51) Int. Cl. G01N 33/53; G01N 33/542; C12M 1/34; B05D 3/00

(52) U.S. Cl. 435/7.9; 435/287.2; 427/2.11

(57) ABSTRACT

The present invention relates to methods for making a stable thin film dried protein composition on a surface that has a protein denaturing capability and related devices. A thin film of a biologically active protein containing solution to be dried is deposited on the support surface wherein the film also contains a buffer that maintains the surface pH between about 5.0 and 9.0 during drying under normal pressures and a saccharide in an amount sufficient to stabilize the protein while drying. An advantage of the present invention is that one can make thin film assay devices, such as proteomic microarrays, using supports that normally would not be useful. These supports include modified, chemically derivatized or coated glass substrates.

### TECHNICAL FIELD

The present invention relates to a novel stable dried protein composition on a surface that has a protein denaturing capability and related methods. A thin film of a biologically active protein containing solution to be dried is deposited on the support surface wherein the film also contains a buffer that maintains the surface pH between about 5.0 and 9.0 during drying under normal pressures and a saccharide in an amount sufficient to stabilize the protein while drying. An advantage of the present invention is that one can make thin film assay devices, such as proteomic microarrays, using supports that normally would not be useful. These supports include modified, chemically derivatized, or coated glass substrates.

### BACKGROUND ART

The problem of stabilizing proteins for bulk lyophilization has been addressed by the prior art.

U.S. Pat. No. 6,238,664 to Hellerbrand et alia discloses a process for reconstituting a lyophilizate formed from an aqueous solution having a protein dissolved therein, being resistant to the formation of protein aggregates and particles under conditions of freezing, thawing, lyophilization, and reconstitution. The solution contains potassium ions and either contains no sodium ions or contains sodium ions such that the ratio of potassium ions to sodium ions in the solution is at least 10:1. The solution is buffered at a pH from 6 to 8 with a potassium phosphate buffer in a concentration of from 10 to 300 mmol/liter in the solution.

U.S. Pat. No. 6,267,958 to Andya et alia discloses a stable lyophilized protein formulation which can be reconstituted with a suitable diluent to generate a high protein antibody concentration reconstituted formulation which is suitable for subcutaneous administration by using a lyoprotectant. A stable isotonic reconstituted formulation comprises an antibody in an amount of about 50 mg/mL to about 400 mg/mL and a diluent, which reconstituted formulation has been prepared from a lyophilized mixture of the antibody and a lyoprotectant (sucrose or trehalose) which prevents or reduces chemical or physical instability of the antibody upon lyophilization and subsequent storage. The molar ratio of lyoprotectant:antibody is about 100-510 mole lyoprotectant:1 mole of antibody. The antibody concentration in the reconstituted formulation is about 2-40 times greater than the antibody concentration in the mixture before lyophilization.

U.S. Pat. No. 6,251,599 to Chen et alia discloses preparations of nucleic acid condensates and compositions containing such condensates. The nucleic acid condensates are in the form of small particles that are stable when subjected to destabilizing conditions such as lyophilizing, freeze-thawing, and prolonged liquid storage. These compositions may be used to deliver nucleic acid to cells. In particular, the related composition comprises a nucleic acid condensed with a polycation in a liquid medium, thereby forming a particle, and at least one excipient selected from the group consisting of a zwitterion, an amorphous cryoprotectant (such as asaccharide, a polyol or a protein.), a crystalline bulking agent, and mixtures thereof The particle increases in size by less than one-fold during storage in the liquid medium for one week at about 2.degree. C. to about 8.degree. C.

The prior art has used trehalose to protect dried products that contains bulk proteins as well as other ingredients.

U.S. Pat. No. 6,005,100 to Mandai et alia discloses a novel desiccant containing a non-reducing anhydrous trehalose as an effective ingredient for the dehydration of food products, pharmaceuticals and cosmetics. Anhydrous trehalose is incorporated into the hydrous matters to convert the anhydrous trehalose into hydrous crystalline trehalose.

U.S. Pat. No. 4,891,319 to Roser discloses a method of protecting proteins or other biological macromolecules against denaturation during drying, comprising subjecting an aqueous system containing the protein or other biological macromolecule to drying at a temperature above freezing in the presence of trehalose in an amount between about 0.05 and 20 weight percent based on the total weight of said aqueous system.

### DISCLOSURE OF THE INVENTION

An object of the present invention is to provide a means for creating thin stable films of dried proteins on support surfaces that can otherwise denature or adversely affect the proteins upon drying (e.g., a deterioration or loss of enzymatic or specific binding activity) at normal ambient temperatures and pressures. With the increasing interest in proteomic microarrays, the art faces a problem with the stability of applying thin films to a microarray support for drying. In particular, the sensitivity and specificity requirements for proteomic microarrays require that little, if any, of the protein present in a dried microarray spot lose its tested biological activity. Unfortunately, this is not the case for the combination of the protein candidates for microarrays and the surfaces to which they are applied. Biological activity losses that are acceptable in other applications are not acceptable in proteomic microarray testing.

The present invention encompasses a novel method for drying proteins in a solution without requiring special conditions (such as lyophilization) and devices created by such methods. A method for producing a thin film dried protein composition comprises three steps. First, one makes a protein containing solution that is to be dried on a surface, preferably a biologically active protein. (For the purposes of the present invention, the term “biologically active” includes any protein that can participate in a specific binding reaction, (such as antibodies, antibody fragments, antigens, antigen fragments), as well as peptides or enzymes.) The solution is made with a buffer that maintains the surface pH between about 5.0 and 9.0 during solution drying and with a saccharide in an amount sufficient to stabilize the protein during solution drying. The selection of the pH should consider the optimal pH for the biologically active protein. One of ordinary skill in the art can appreciate that, for example, some enzymes prefer a more alkaline pH. Second, one applies a thin film of the solution to a support having the surface for depositing, the surface having a protein denaturing capability. Third, one allows the thin film of protein containing solution to dry on the support surface under normal pressures.

The present method enables one to make stable thin film dried protein compositions. Such films can be incorporated into protein analytical devices. Of particular interest are proteomic microarrays.

### BRIEF DESCRIPTION OF THE DRAWINGS

FIG. 1 is a graph showing the retention of specific signal from proteomic microarrays made with and without a saccharide.

FIG. 2 is a graph showing the retention of specific signal from proteomic microarrays made with and without a saccharide.

FIG. 3 is a graph showing the retention of specific signal from proteomic microarrays made with different saccharides.

FIG. 4 is a graph showing the effect on long-term shelf life for specific signal from a proteomic microarray made according to the present invention.

### BEST MODES FOR CARRYING OUT THE INVENTION

In preferred embodiments, the present invention relates to stable thin film dried protein composition. Suitable buffers for use in the present invention include Tris, Mes, HEPES, carbonates, or phosphates. Suitable saccharides for use in the present invention include trehalose, sucrose, maltose, lactose, mannitol, xylitol, dextran, glucose, xylose, fructose, maltodextrin, or glucuronic acid, preferably at concentrations of up to 10% w/v, typically about 1% to 5% w/v.

In some preferred embodiments, one can also include conventional surfactants in known concentrations in the protein containing solution prior to drying. Applicants believe that these surfactants are used for film wetting purposes. Suitable surfactants include typical sodium salts of long chain carbonyl linked sulfonic acids (sulfonates) or sulfuric acids (sulfates), e.g., sodium olefin sulfonate or sodium dodecyl sulfate) used at wetting concentrations, typically less than 2% w/v.

In some preferred embodiments, one can also include alkyl alcohols in the protein containing solution prior to drying, either alone or in combination with a surfactant. These alkyl alcohols provide additional stability, possibly by accelerating protein association with the drying surface.. Suitable alkyl alcohols include methanol, ethanol, and propanol at concentrations of up to 15% v/v, preferably about 1% v/v to about 10% v/v.

The present dried thin protein films can be incorporated into conventional proteomic microarrays. Thus, a plurality of discretely spaced thin dried protein films can be placed onto a conventional microarray support surface. Suitable surfaces include conventionally available polymers, glass, metals or alloys, and coated glass (including metal or polymer coatings, such as either gold coatings on glass or a microporous membrane surface such as nitrocellulose, nylon, polyethersulfones, or polyvinylidene fluoride on glass). Alternatively, the support surface can be treated so as to provide a binding site for the protein. The retention of biological activity is important for microarrays because such minute quantities are retained on the device surface. Protein denaturation due to surface interactions can have a significant limiting factor on the sensitivity of the device, particularly in view of the small volumes deposited on the microarray. One uses these arrays by generating and measuring either an isotopic or an electromagnetic radiation signal (typically fluorogenic or chromagenic in nature) from any attached protein signal component.

### Performance

The performance of the present invention was assessed in the following manner. A solution was made consisting of a buffer, trehalose or another saccharide, in some cases an alcohol, and in some cases also a surfactant, as noted below. Specifically, the buffer comprised 0.1N sodium bicarbonate (pH 7.4) and 0.1% w/v sodium olefin sulfonate, and 5% trehalose or another saccharide, also as noted below. This solution was used to dissolve several antibody proteins at concentrations ranging from 0.1 mg/ml to 2.0 mg/ml.

Small volumes of these solutions (0.7 nl or 1 nl containing 1.5 mg/ml of protein) were spotted (arrayed) onto various surfaces using robotic manipulation. The resulting arrays were dried at room temperature and normal pressure. This manipulation resulted in thin circular films of dried protein and antibodies. Each circle was less than 100 microns in diameter and about 1 to 5 microns thick.

Coated glass was the support used for these preferred embodiments. The glass was coated with a microporous material, such as cellulose nitrate ester (approximately 15 micrometers in thickness).

Activity of the arrayed proteins and antibodies in the thin film was determined as follows. In one test, the surfaces with the films were exposed to a solution containing a protein with an affinity to the arrayed, dried antibody. The protein was linked covalently to a fluorescent reporter molecule. After exposure to the labeled protein, the surfaces were washed and imaged by digital confocal laser-based microscopy. The intensity of the fluorescent signal was proportional to the amount of protein present, which in turn indicated how active the dried thin film antibody was.

In a second test, the dried thin films were exposed to a solution containing proteins with affinities to the dried antibody. Again, the films were exposed to a solution phase antibody with an affinity to the first protein. The second antibody was linked covalently to a small molecule, biotin. Finally, the films were exposed to a biotin-affinity solution of streptavidin linked to a fluorescent reporter molecule. These films were imaged by digital confocal laser based microscopy. The intensity of the fluorescent signal was proportional to the amount of protein present, which in turn indicated how active the dried thin film antibody was.

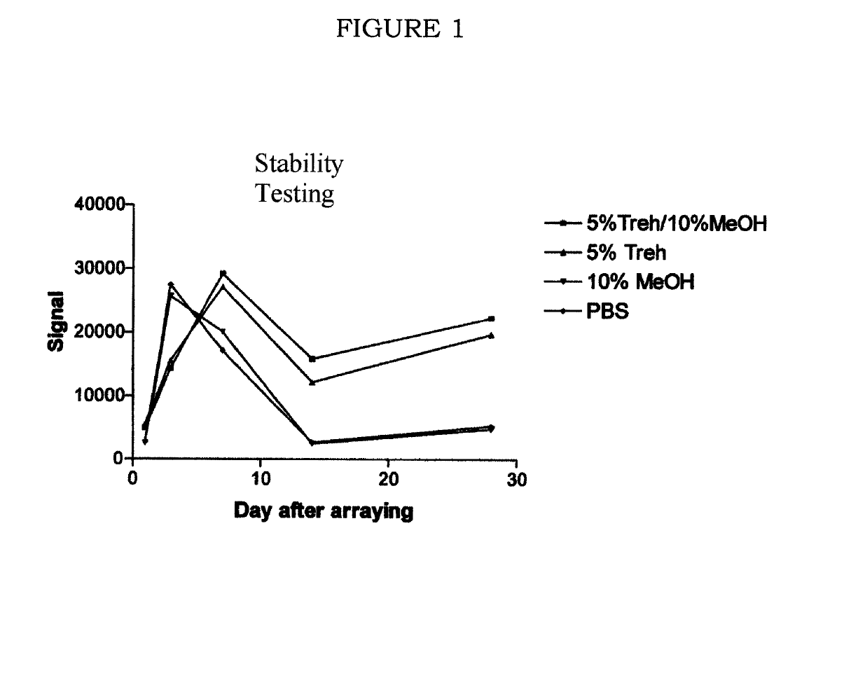

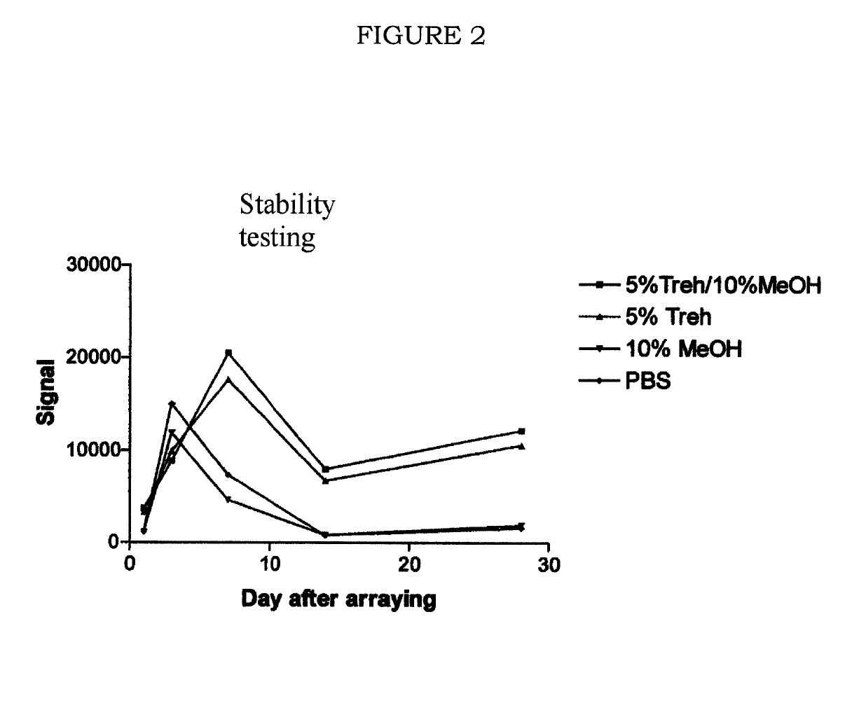

As seen in FIGS. 1 and 2, the results of this testing demonstrates that thin films made with the present buffer composition, preserved the binding activity of the arrayed antibodies throughout the drying process and over time. In FIGS. 1 and 2, a series of compositions were tested including a PBS/antibody buffer solution, a PBS/antibody/10% methanol solution, a PBS antibody/5% trehalose solution, and a PBS/5% trehalose/10% methanol solution. Surfactant was present on the coated surface in a concentration of about 0.1% w/v. (One should note that FIG. 2 is a repeat of the testing of FIG. 1 with the protein concentration lowered from 1.5 mg/ml to 0.375 mg/ml.) Spotted and dried solution spots were tested for shelf life, i.e., the retention of biological activity, in this case, a specific binding reaction. Antibodies arrayed without the saccharide-containing buffer quickly lost binding activity, even in the presence of the alcohol.

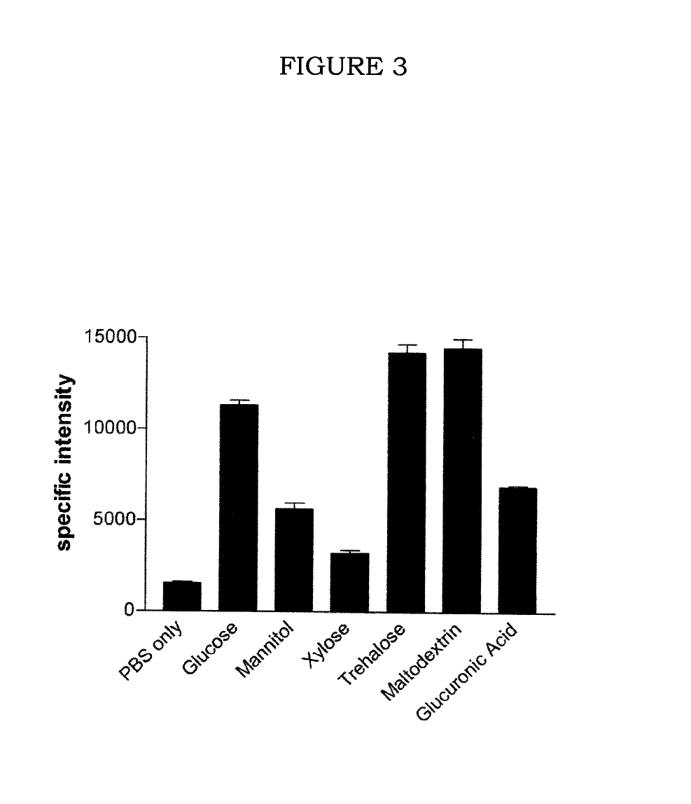

In FIG. 3, a series of compositions were tested including a PBS/5% trehalose/10% methanol solution containing 1.5 mg/ml of antibody protein. The difference amongst the solutions was the saccharide used, namely, glucose, mannitol, xylose, trehalose, maltodextrin, and glucuronic acid. Spotted and dried solution spots were tested for shelf life, i.e., the retention of biological activity, in this case, a specific binding reaction. While some saccharides delivered a higher specific signal than others, all delivered a signal at least twice that of the control solution which did not contain any saccharide.

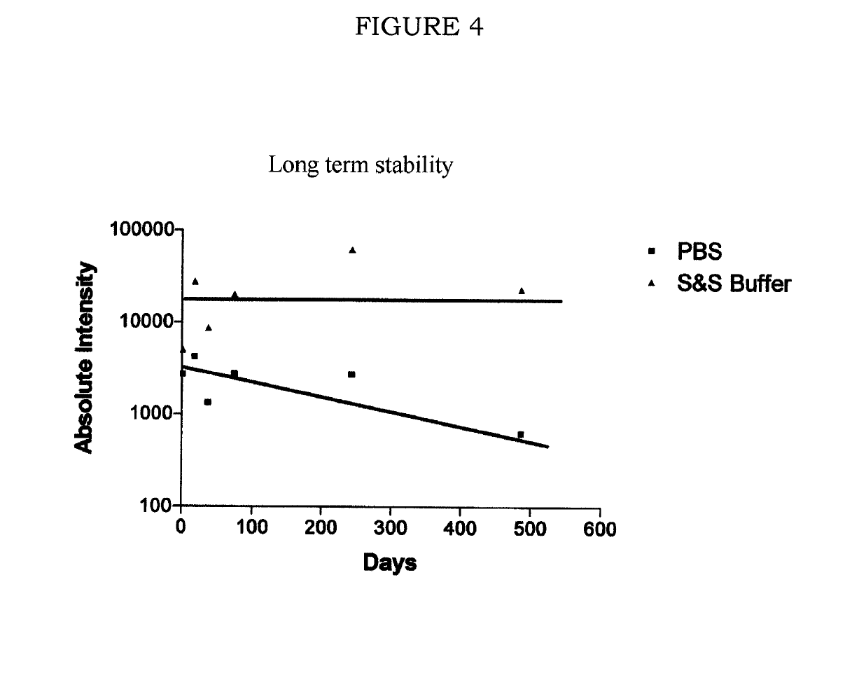

In FIG. 4, an accelerated shelf life study was done on the influence of the present invention. A comparison was made between a control solution containing a 1.5 mg/ml antibody protein solution and a PBS/5% trehalose/10% methanol solution. The dried spotted microarrays were held at 37 degrees C. for up to 120 days which, samples being periodically withdrawn and tested for signal intensity. By using an elevated temperature, one can extrapolate anticipated shelf life for a dried protein composition. One can clearly see that the present thin protein film maintained signal, and hence binding activity, for over a year. However, the control microarray lost substantial and significant signal over the same time period.

These results indicate that the present invention does provide for stable thin films under conditions that ordinarily would affect protein activity.

The ordinarily skilled artisan can appreciate that the present invention can incorporate any number of the preferred features described above.

All publications or unpublished patent applications mentioned herein are hereby incorporated by reference thereto.

Other embodiments of the present invention are not presented here which are obvious to those of ordinary skill in the art, now or during the term of any patent issuing from this patent specification, and thus, are within the spirit and scope of the present invention.

We claim:

1. A thin film dried protein composition comprising:
a) a support having a surface for depositing a solution to be dried that contains a biologically active protein, the surface having a protein denaturing capability; and
b) a thin film of dried protein on the support surface wherein the film also contains a dried buffer that maintained the surface pH between about 5.0 and 9.0 during solution drying and a saccharide in an amount sufficient to stabilize the protein during solution drying.

2. The thin film dried protein composition of claim 1 wherein the saccharide comprises trehalose, sucrose, maltose, lactose, xylitol, dextran, fructose, mannitol, glucose, xylose, maltodextrin or glucuronic acid.

3. The thin film dried protein composition of claim 1 wherein the solution also contains a surfactant.

4. The thin film dried protein composition of claim 1 wherein the solution also contains an alkyl alcohol.

5. The thin film dried protein composition of claim 3 wherein the solution also contains an alkyl alcohol.

6. The thin film dried protein composition of claim 1 wherein the protein has biological activity and participates in a specific binding reaction.

7. The thin film dried protein composition of claim 6 wherein the protein comprises antibodies, antibody fragments, antigens, antigen fragments, peptides, or enzymes.

8. The thin film dried protein composition of claim 1 wherein the support is glass having a microporous membrane surface.

9. The thin film dried protein composition of claim 1 wherein the microporous surface comprises nitrocellulose, nylon, polyethersulfones, or polyvinylidene fluoride.

10. A thin film dried protein analytical device comprising:
a) a support having a surface for depositing a solution to be dried that contains a biologically active protein, the surface having a protein denaturing capability;
b) a plurality of discretely placed thin films of dried protein on the support surface wherein the films also contain a dried buffer that maintained the surface pH between about 5.0 and 9.0 during solution drying and a saccharide in an amount sufficient to stabilize the protein during solution drying, wherein the protein in each film is capable of participating in a specific binding reaction.

11. A method for producing a thin film dried protein composition comprising
a) making a biologically active protein containing solution to be dried on a surface with a buffer that maintains the surface pH between about 5.0 and 9.0 during solution drying and with a saccharide in an amount sufficient to stabilize the protein during solution drying, the surface having a protein denaturing capability;
b) applying a thin film of the solution to a support having the surface for depositing, the surface having a protein denaturing capability; and
c) allowing the thin film of protein containing solution to dry on the support surface under normal pressures.

12. A method for analyzing a thin film dried protein microarray comprising:
a) attaching a plurality of sample biologically active proteins to the device of claim 10, each at a discrete thin film site;
b) attaching at least one protein signal component to at least one of the attached sample proteins in the microarray; and
c) generating and measuring an electromagnetic radiation signal from any attached protein signal component.

13. The method of claim 12 wherein the signal component is a fluorogen or a chromagen.

14. The method of claim 12 wherein the signal is fluorescence, near ultra-violet light, near infra-red light, or visible light.
